(19) United States
(12) Patent Application Publication
(10) Pub. No.: US 2009/0236591 A1
(43) Pub. Date: Sep. 24, 2009

(54) N,N'-BIS(FLUOROPHENYLALKYL)-SUBSTITUTED PERYLENE-3,4:9,10-TETRACARBOXIMIDES, AND THE PREPARATION AND USE THEREOF

(75) Inventors: Martin Konemann, Mannheim (DE); Rudiger Schmidt, Paderborn (DE); Frank Wurthner, Hoechberg (DE); Zhenan Bao, Stanford, CA (US); Joon Hak Oh, Palo Alto, CA (US)

Correspondence Address: OBLON, SPIVAK, MCCLELLAND MAIER & NEUSTADT, P.C., 1940 DUKE STREET, ALEXANDRIA, VA, 22314, US

(73) Assignees: BASF SE, Ludwigshafen (DE); The Board of Trust. of the Lelnd Stanf. Jun. Univ., Palo Alto, CA (US)

(21) Appl. No.: 12/406,448

(22) Filed: Mar. 18, 2009

Related U.S. Application Data
(60) Provisional application No. 61/037,863, filed on Mar. 19, 2008.

(51) Int. Cl. H01L 51/30; C07D 471/06; C07D 493/06

(52) U.S. Cl. 257/40; 546/37; 549/232; 257/E51.024

(57) ABSTRACT

The present invention relates to N,N′-bis(fluorophenylalkyl)-substituted perylene-3,4:9,10-tetracarboximides, their preparation and their use as charge transport materials, exciton transport materials or emitter materials.

### SUBJECT MATTER OF THE INVENTION

The present invention relates to N,N′-bis(fluorophenylalkyl)-substituted perylene-3,4:9,10-tetracarboximides, their preparation and their use as charge transport materials, exciton transport materials or emitter materials.

### STATE OF THE ART

It is expected that, in the future, not only the classical inorganic semiconductors but increasingly also organic semiconductors based on low molecular weight or polymeric materials will be used in many sectors of the electronics industry. In many cases, these organic semiconductors have advantages over the classical inorganic semiconductors, for example better substrate compatibility and better processibility of the semiconductor components based on them. They allow processing on flexible substrates and enable their interface orbital energies to be adjusted precisely to the particular application sector by the methods of molecular modeling. The significantly reduced costs of such components have brought a renaissance to the field of research of organic electronics. “Organic electronics” is concerned principally with the development of new materials and manufacturing processes for the production of electronic components based on organic semiconductor layers. These include in particular organic field-effect transistors (OFETs) and organic light-emitting diodes (OLEDs), and photovoltaics. Great potential for development is ascribed to organic field-effect transistors, for example in memory elements and integrated optoelectronic devices. Organic light-emitting diodes (OLEDs) exploit the property of materials of emitting light when they are excited by electrical current. OLEDs are particularly of interest as alternatives to cathode ray tubes and liquid-crystal displays for producing flat visual display units. Owing to the very compact design and the intrinsically lower power consumption, devices which comprise OLEDs are suitable especially for mobile applications, for example for applications in cellphones, laptops, etc. Great potential for development is also ascribed to materials which have maximum transport widths and high mobilities for light-induced excited states (high exciton diffusion lengths) and which are thus advantageously suitable for use as an active material in so-called excitonic solar cells. It is generally possible with solar cells based on such materials to achieve very good quantum yields.

Min-Min Shi et al. describe, in Acta Chimica Sinica, Vol.64, 2006, No. 8, p. 721-726, the electron mobilities of N,N′-bisperfluorophenyl-3,4:9,10-perylenetetracarboximide and N,N′-bis(1,1-dihydroperfluorooctyl)-3,4:9,10-perylenetetracarboximide. The electron mobilities of these compounds are still in need of improvement with regard to use as organic field-effect transistors and in organic photovoltaics. A possible use in excitonic solar cells is not described.

Z. Bao et al. describe, in Chem. Mater. 2007, 19, 816-824, the use of fluorinated derivatives of perylenediimides as n-semiconductors in thin-film transistors (TFTs). In this case, perylenediimides in which the imide nitrogen atoms bear fluorinated aryl radicals are used.

WO 2007/074137 describes compounds of the general formula (A)

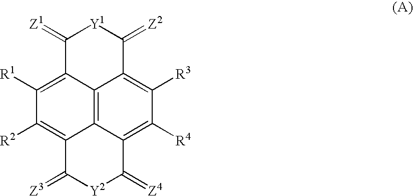

where

at least one of the R¹, R², R³ and R⁴ radicals is a substituent which is selected from Br, F and CN,

Y¹ is O or NR^{a}, where R^{a} is hydrogen or an organyl radical,

Y² is O or NR^{b}, where R^{b} is hydrogen or an organyl radical,

Z¹ and Z² are each independently O or NR^{c}, where R^{c} is an organyl radical,

Z³ and Z⁴ are each independently O or NR^{d}, where R^{d} is an organyl radical,

where, in the case that Y¹ is NR^{a} and at least one of the Z¹ and Z² radicals is NR^{c}, R^{a} with one R^{c} radical may also together be a bridging group having 2 to 5 atoms between the flanking bonds, and

where, in the case that Y² is NR^{b} and at least one of the Z³ and Z⁴ radicals is NR^{d}, R^{b} with one R^{d} radical may also together be a bridging group having 2 to 5 atoms between the flanking bonds,

and their use as n-semiconductors in organic field-effect transistors.

WO 2007/093643 describes the use of compounds of the general formula (B)

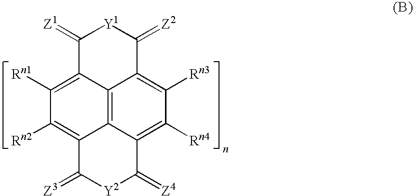

where

n is 2, 3 or 4,

at least one of the Rⁿ¹, Rⁿ², Rⁿ³ and Rⁿ⁴ radicals is fluorine,

if appropriate, at least one further Rⁿ¹, Rⁿ², Rⁿ³ and Rⁿ⁴ radical is a substituent which is independently selected from Cl and Br, and the remaining radicals are each hydrogen,

Y¹ is O or NR^{a} where R^{a} is hydrogen or an organyl radical,

Y² is O or NR^{b} where R^{b} is hydrogen or an organyl radical,

Z¹, Z², Z³ and Z⁴ are each O,

where, in the case that Y¹ is NR^{a}, it is also possible for one of the Z¹ and Z² radicals to be NR^{c}, where the R^{a} and R^{c} radicals together are a bridging group having from 2 to 5 atoms between the flanking bonds, and

where, in the case that Y² is NR^{b}, it is also possible for one of the Z³ and Z⁴ radicals to be NR^{d}, where the R^{b} and R^{d} radicals together are a bridging group having from 2 to 5 atoms between the flanking bonds,

as semiconductors, especially as n-semiconductors, in organic electronics, especially for organic field-effect transistors, solar cells and organic light-emitting diodes.

The unpublished U.S. 60/945,704 describes the use of compounds of the general formula (C)

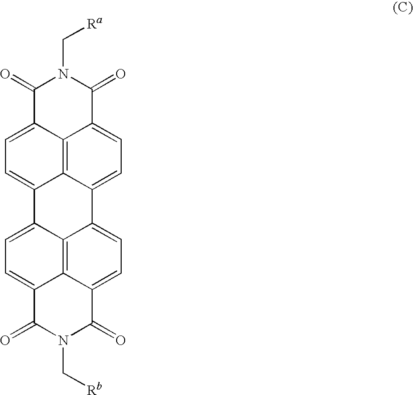

where

R^{a} and R^{b} are each independently perfluoro-C₂-C₄-alkyl

as charge transport materials or exciton transport materials.

It has now been found that, surprisingly, N,N′-bis(fluorophenylalkyl)-substituted perylene-3,4:9,10-tetracarboximides are particularly advantageously suitable as charge transport materials, exciton transport materials or emitter materials. They are notable especially as n-semiconductors with high charge mobilities. Furthermore, the resulting components are air-stable.

### SUMMARY OF THE INVENTION

The invention firstly provides a compound of the general formula (I)

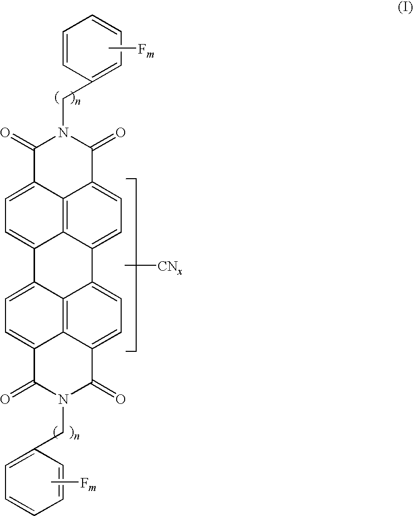

in which

m is 2, 3, 4 or 5,

n is 1, 2 or 3, and

x is 0 or 2.

The invention further provides a process for preparing a compound of the general formula I as defined above, in which a compound of the general formula II

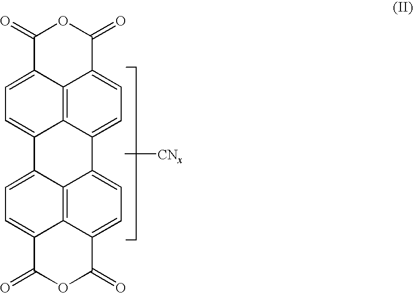

in which x is 0 or 2

is reacted with an amine of the general formula III

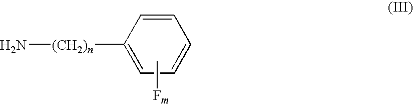

in which

n is 1, 2 or 3, and

m is 2, 3, 4 or 5.

The invention further provides for the use of compounds of the general formula I as defined above as charge transport materials, exciton transport materials or emitter materials.

The invention further provides for the use of compounds of the general formula I as defined above as semiconductor material in organic electronics.

The invention further provides for the use of compounds of the general formula I as defined above as active material in organic photovoltaics (OPVs), especially as exciton transport material in excitonic solar cells.

The invention further provides an organic field-effect transistor (OFET) comprising a substrate having at least one gate structure, a source electrode and a drain electrode and at least one compound of the formula I as defined above as an n-semiconductor.

The invention further provides a substrate comprising a multitude of organic field-effect transistors, wherein at least some of the field-effect transistors comprise at least one compound of the formula I as defined above as an n-semiconductor. The invention also provides a semiconductor unit comprising at least one such substrate.

The invention further provides an organic light-emitting diode (OLED) comprising at least one compound of the formula I as defined above.

The invention further provides an organic solar cell comprising at least one compound of the formula I as defined above.

### DESCRIPTION

In the compound of the general formula I, m is preferably 5.

In the compound of the general formula I, n is preferably 2.

In the compound of the general formula I, x is preferably 0.

In the compounds of the general formula I, the fluorophenylalkyl groups are of the formula

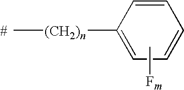

in which # represents the bonding site to an imide nitrogen atom, selected from

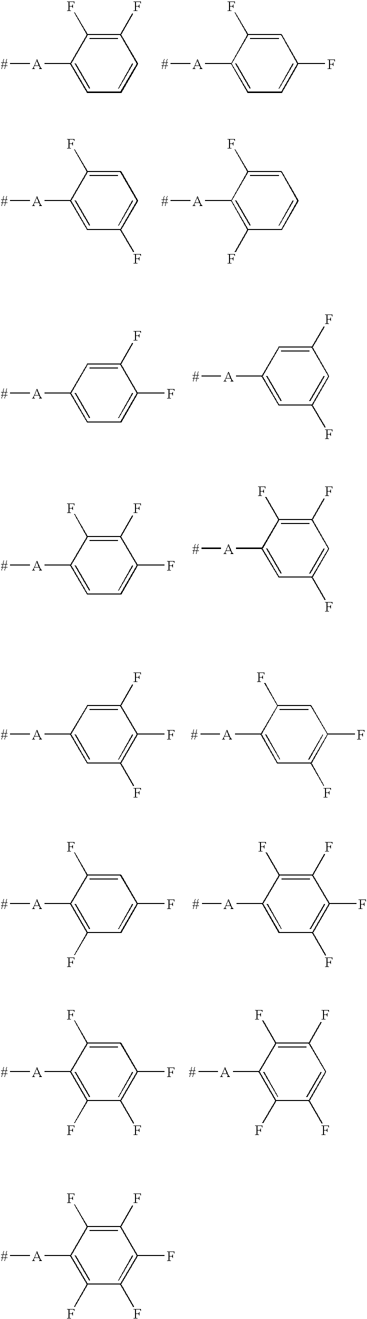

in which # represents the bonding site to an imide nitrogen atom, and

A is CH₂, (CH₂)₂ or (CH₂)₃.

In the compounds of the general formula I, the fluorophenylalkyl groups are particularly preferably selected from groups of the formulae

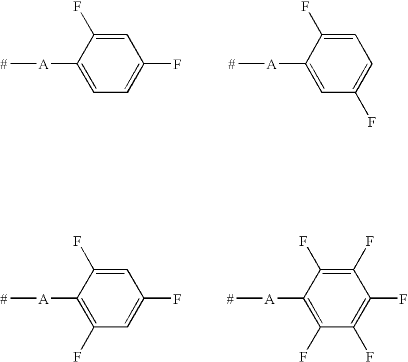

in which # represents the bonding site to an imide nitrogen atom, and

A is CH₂, (CH₂)₂ or (CH₂)₃.

A in the formulae listed above is especially (CH₂)₂.

In the compounds of the general formula I, the fluorophenylalkyl groups of the formula

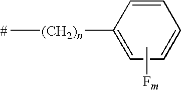

are preferably both

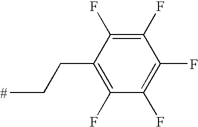

in which # represents the bonding site to an imide nitrogen atom.

Some preferred compounds of the formula I are depicted below by way of example:

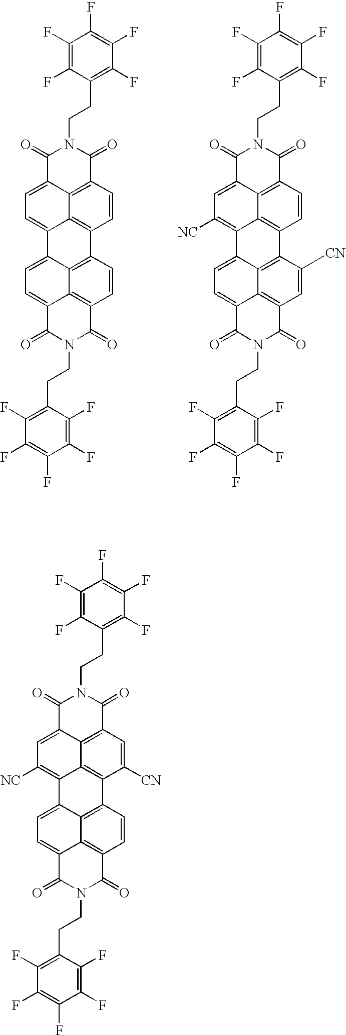

To prepare the compounds of the general formula I, a compound of the general formula II

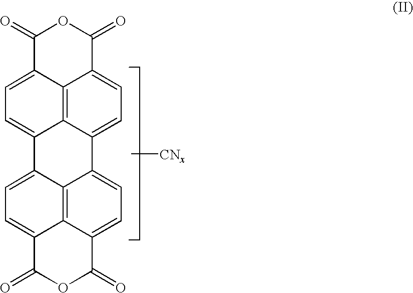

in which x is 0 or 2

can be reacted with an amine of the general formula III

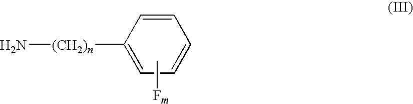

in which

n is 1, 2 or 3, and

m is 2, 3, 4 or 5.

The imidation of carboxylic anhydride groups is known in principle. Preference is given to reacting the dianhydride with the primary amine in the presence of a polar aprotic solvent. Suitable polar aprotic solvents are nitrogen heterocycles, such as pyridine, pyrimidine, quinoline, isoquinoline, quinaldine, N-methylpiperidine, N-methylpiperidone and N-methylpyrrolidone.

The reaction can be undertaken in the presence of an imidation catalyst. Suitable imidation catalysts are organic and inorganic acids, for example formic acid, acetic acid, propionic acid and phosphoric acid. Suitable imidation catalysts are also organic and inorganic salts of transition metals such as zinc, iron, copper and magnesium. These include, for example, zinc acetate, zinc propionate, zinc oxide, iron(II)acetate, iron(III)chloride, iron(II)sulfate, copper(II)acetate, copper(II)oxide and magnesium acetate. The amount of the imidation catalyst used is preferably from 1 to 80% by weight, more preferably from 5 to 50% by weight, based on the total weight of the compound to be imidated.

The molar ratio of amine to dianhydride is preferably from about 2:1 to 10:1, more preferably from 2.2:1 to 8:1.

The reaction temperature is generally from about 20° C. to 250° C., preferably from 80° C. to 200° C. Aliphatic and cycloaliphatic amines are reacted preferably within a temperature range of from about 60° C. to 100° C. Aromatic amines are reacted preferably within a temperature range of from about 120° C. to 160° C.

Preference is given to effecting the reaction under a protective gas atmosphere, for example nitrogen.

The reaction can be effected under standard pressure or, if desired, under elevated pressure. A suitable pressure range is in the range from about 0.8 to 10 bar. In the case of use of volatile amines (boiling point about ≦180° C.), preference is given to working under elevated pressure.

In general, the resulting diimides can be used for the subsequent reactions without further purification. For use of the products as semiconductors, it may, however, be advantageous to subject the products to a further purification. Examples include column chromatography processes, in which the products are preferably dissolved in a halogenated hydrocarbon, such as methylene chloride, chloroform or tetrachloro-ethane, an aromatic such as toluene or xylene, or a mixture thereof, and subjected to a separation or filtration on silica gel. Finally, the solvent is removed.

The amines of the general formula III can be provided by customary methods known to those skilled in the art. They are provided, for example, proceeding from nitriles of the formula IV

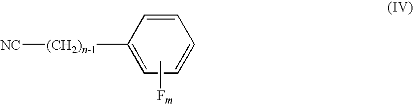

by reduction, for example with hydrogen in the presence of ammonia or with complex hydrides such as LiAlH₄, NaBH₄, etc. Corresponding nitriles, such as 2,3,4,5,6-pentafluorophenylacetonitrile, are in many cases commercially available or preparable via standard methods.

The inventive compounds (I) are particularly advantageously suitable as organic semiconductors. They generally function as n-semiconductors. When the compounds of the formula (I) used in accordance with the invention are combined with other semiconductors and the position of the energy levels causes the other semiconductors to function as n-semiconductors, the compounds (I) can also function as p-semiconductors in exceptional cases.

The compounds of the formula (I) are notable for their air stability.

The compounds of the formula (I) possess a high charge transport mobility and/or have a high on/off ratio. They are particularly advantageously suitable for organic field-effect transistors (OFETs).

The inventive compounds are advantageously suitable for producing integrated circuits (ICs) for which the n-channel MOSFETs (metal oxide semiconductor field-effect transistors (MOSFETs)) customary to date are used. These are then CMOS-like semiconductor units, for example for microprocessors, microcontrollers, static RAM, and other digital logic units.

For the production of semiconductor materials, the inventive compounds of the formula (I) can be processed further by one of the following processes: printing (offset, flexographic, gravure, screen, inkjet, electrophotography), evaporation, laser transfer, photolithography, dropcasting. They are suitable especially for use in displays (especially large-area and/or flexible displays) and RFID tags.

The inventive compounds are also particularly suitable as fluorescence emitters in OLEDs, in which they are excited either by electroluminescence or by a corresponding phosphorescence emitter via Förster energy transfer (FRET).

The inventive compounds of the formula (I) are also particularly suitable in displays which, based on an electrophoretic effect, switch colors on and off via charged pigment dyes. Such electrophoretic displays are described, for example, in US 2004/0130776.

The invention further provides organic field-effect transistors comprising a substrate having at least one gate structure, a source electrode and a drain electrode and at least one compound of the formula I as defined above as an n-semiconductor. The invention further provides substrates comprising a multitude of organic field-effect transistors, wherein at least some of the field-effect transistors comprise at least one compound of the formula I as defined above as an n-semiconductor. The invention also provides semiconductor units which comprise at least one such substrate.

A specific embodiment is a substrate with a pattern (topography) of organic field-effect transistors, wherein each transistor comprises
an organic semiconductor disposed on the substrate;
a gate structure for controlling the conductivity of the conductive channel; and
conductive source and drain electrodes at the two ends of the channel,

wherein the organic semiconductor consists of at least one compound of the formula (I) or comprises a compound of the formula (I). In addition, the organic field-effect transistor generally comprises a dielectric.

A further specific embodiment is a substrate having a pattern of organic field-effect transistors, wherein each transistor forms an integrated circuit or is part of an integrated circuit and at least some of the transistors comprise at least one compound of the formula (I).

Suitable substrates are in principle the materials known for this purpose. Suitable substrates comprise, for example, metals (preferably metals of groups 8, 9, 10 or 11 of the Periodic Table, such as Au, Ag, Cu), oxidic materials (such as glass, quartz, ceramics, SiO₂), semiconductors (e.g. doped Si, doped Ge), metal alloys (for example based on Au, Ag, Cu, etc.), semiconductor alloys, polymers (e.g. polyvinyl chloride, polyolefins such as polyethylene and polypropylene, polyesters, fluoropolymers, polyamides, polyimides, polyurethanes, polyalkyl (meth)acrylates, polystyrene and mixtures and composites thereof), inorganic solids (e.g. ammonium chloride), paper and combinations thereof. The substrates may be flexible or inflexible, and have a curved or planar geometry, depending on the desired use.

A typical substrate for semiconductor units comprises a matrix (for example a quartz or polymer matrix) and, optionally, a dielectric top layer.

Suitable dielectrics are SiO₂, polystyrene, poly-α-methylstyrene, polyolefins (such as polypropylene, polyethylene, polyisobutene), polyvinylcarbazole, fluorinated polymers (e.g. Cytop, CYMM), cyanopullulans, polyvinylphenol, poly-p-xylene, polyvinyl chloride, or polymers crosslinkable thermally or by atmospheric moisture. Specific dielectrics are “self-assembled nanodielectrics”, i.e. polymers which are obtained from monomers comprising SiCl functionalities, for example Cl₃SiOSiCl₃, Cl₃Si—(CH₂)₆—SiCl₃, Cl₃Si—(CH₂)₁₂—SiCl₃, and/or which are crosslinked by atmospheric moisture or by addition of water diluted with solvents (see, for example, Faccietti Adv. Mat. 2005, 17, 1705-1725). Instead of water, it is also possible for hydroxyl-containing polymers such as polyvinyl-phenol or polyvinyl alcohol or copolymers of vinylphenol and styrene to serve as crosslinking components. It is also possible for at least one further polymer to be present during the crosslinking operation, for example polystyrene, which is then also crosslinked (see Facietti, US patent application 2006/0202195).

The substrate may additionally have electrodes, such as gate, drain and source electrodes of OFETs, which are normally localized on the substrate (for example deposited onto or embedded into a nonconductive layer on the dielectric). The substrate may additionally comprise conductive gate electrodes of the OFETs, which are typically arranged below the dielectric top layer (i.e. the gate dielectric).

In a specific embodiment, a gate insulating layer is present on at least part of the substrate surface. The gate insulating layer comprises at least one insulator which is preferably selected from inorganic insulators such as SiO₂, SiN, etc., ferroelectric insulators such as Al₂O₃, Ta₂O₅, La₂O₅, TiO₂, Y₂O₃, etc., organic insulators such as polyimides, benzocyclobutene (BCB), polyvinyl alcohols, polyacrylates, etc., and combinations thereof.

Suitable materials for source and drain electrodes are in principle electrically conductive materials. These include metals, preferably metals of groups 8, 9, 10 or 11 of the Periodic Table, such as Pd, Au, Ag, Cu, Al, Ni, Cr, etc. Also suitable are conductive polymers such as PEDOT (=poly(3,4-ethylenedioxythiophene)); PSS (=poly(styrenesulfonate)), polyaniline, surface-modified gold, etc. Preferred electrically conductive materials have a specific resistance of less than 10⁻³ ohm×meter, preferably less than 10⁻⁴ ohm×meter, especially less than 10⁻⁶ or 10⁻⁷ ohm×meter.

In a specific embodiment, drain and source electrodes are present at least partly on the organic semiconductor material. It will be appreciated that the substrate may comprise further components as used customarily in semiconductor materials or ICs, such as insulators, resistors, capacitors, conductor tracks, etc.

The electrodes may be applied by customary processes, such as evaporation, lithographic processes or another structuring process.

The semiconductor materials may also be processed with suitable auxiliaries (polymers, surfactants) in disperse phase by printing.

In a preferred embodiment, the deposition of at least one compound of the general formula I (and if appropriate further semiconductor materials) is carried out by a gas phase deposition process (physical vapor deposition, PVD). PVD processes are performed under high-vacuum conditions and comprise the following steps: evaporation, transport, deposition. It has been found that, surprisingly, the compounds of the general formula I are suitable particularly advantageously for use in a PVD process, since they essentially do not decompose and/or form undesired by-products. The material deposited is obtained in high purity. In a specific embodiment, the deposited material is obtained in the form of crystals or comprises a high crystalline content. In general, for the PVD, at least one compound of the general formula I is heated to a temperature above its evaporation temperature and deposited on a substrate by cooling below the crystallization temperature. The temperature of the substrate in the deposition is preferably within a range from about 20 to 250° C., more preferably from 50 to 200° C.

The resulting semiconductor layers generally have a thickness which is sufficient for ohmic contact between source and drain electrodes. The deposition can be effected under an inert atmosphere, for example under nitrogen, argon or helium.

The deposition is effected typically at ambient pressure or under reduced pressure. A suitable pressure range is from about 10⁻⁷ to 1.5 bar.

The compound of the formula (I) is preferably deposited on the substrate in a thickness of from 10 to 1000 nm, more preferably from 15 to 250 nm. In a specific embodiment, the compound of the formula I is deposited at least partly in crystalline form. For this purpose, especially the above-described PVD process is suitable. Moreover, it is possible to use previously prepared organic semiconductor crystals. Suitable processes for obtaining such crystals are described by R. A. Laudise et al. in “Physical Vapor Growth of Organic Semi-Conductors”, Journal of Crystal Growth 187 (1998), pages 449-454, and in “Physical Vapor Growth of Centimeter-sized Crystals of α-Hexa-thiophene”, Journal of Crystal Growth 1982 (1997), pages 416-427, which are incorporated here by reference.

The compounds of the general formula (I) can also advantageously be processed from solution. In that case, the deposition onto a substrate of at least one compound of the general formula (I) (and if appropriate further semiconductor materials) is effected, for example, by spin-coating. The compounds of the formula (I) are also suitable for producing semiconductor elements, especially OFETs, by a printing process. It is possible for this purpose to use customary printing processes (inkjet, flexographic, offset, gravure; intaglio printing, nanoprinting). Preferred solvents for the use of compounds of the formula (I) in a printing process are aromatic solvents such as toluene, xylene, etc. It is also possible to add thickening substances, such as polymers, for example polystyrene, etc., to these “semiconductor inks”. In this case, the dielectrics used are the aforementioned compounds.

In a specific embodiment, the inventive field-effect transistor is a thin-film transistor (TFT). In a customary construction, a thin-film transistor has a gate electrode disposed on the substrate, a gate insulation layer disposed thereon and on the substrate, a semiconductor layer disposed on the gate insulation layer, an ohmic contact layer on the semiconductor layer, and a source electrode and a drain electrode on the ohmic contact layer.

In a preferred embodiment, the surface of the substrate, before the deposition of at least one compound of the general formula (I) (and if appropriate of at least one further semiconductor material), is subjected to a modification. This modification serves to form regions which bind the semiconductor materials and/or regions on which no semiconductor materials can be deposited. The surface of the substrate is preferably modified with at least one compound (C1) which is suitable for binding to the surface of the substrate and to the compounds of the formula (I). In a suitable embodiment, a portion of the surface or the complete surface of the substrate is coated with at least one compound (C1) in order to enable improved deposition of at least one compound of the general formula (I) (and if appropriate further semiconductive compounds). A further embodiment comprises the deposition of a pattern of compounds of the general formula (C1) on the substrate by a corresponding production process. These include the mask processes known for this purpose and so-called “patterning” processes, as described, for example, in U.S. Ser. No. 11/353934, which is incorporated here fully by reference.

Suitable compounds of the formula (C1) are capable of a binding interaction both with the substrate and with at least one semiconductor compound of the general formula I. The term “binding interaction” comprises the formation of a chemical bond (covalent bond), ionic bond, coordinative interaction, van der Waals interactions, e.g. dipole-dipole interactions etc., and combinations thereof. Suitable compounds of the general formula (C1) are:
silanes, phosphonic acids, carboxylic acids, hydroxamic acids, such as alkyltrichlorosilanes, e.g. n-octadecyltrichlorosilane; compounds with trialkoxysilane groups, e.g. alkyltrialkoxysilanes such as n-octadecyltrimethoxysilane, n-octadecyltriethoxysilane, n-octadecyltri(n-propyl)oxysilane, n-octadecyltri(iso-propyl)oxysilane; trialkoxyaminoalkylsilanes such as triethoxyaminopropylsilane and N-[(3-triethoxysilyl)propyl]ethylenediamine; trialkoxyalkyl 3-glycidyl ether silanes such as triethoxypropyl 3-glycidyl ether silane; trialkoxyallylsilanes such as allyltrimethoxysilane; trialkoxy(isocyanatoalkyl)silanes; trialkoxysilyl(meth)-acryloyloxyalkanes and trialkoxysilyl(meth)acrylamidoalkanes such as 1-tri-ethoxysilyl-3-acryloyloxypropane.
amines, phosphines and sulfur-comprising compounds, especially thiols.

The compound (C1) is preferably selected from alkyltrialkoxysilanes, especially n-octadecyltrimethoxysilane, n-octadecyltriethoxysilane; hexaalkyldisilazanes, and especially hexamethyldisilazane (HMDS); C₈-C₃₀-alkylthiols, especially hexadecane-thiol; mercaptocarboxylic acids and mercaptosulfonic acids, especially mercaptoacetic acid, 3-mercaptopropionic acid, mercaptosuccinic acid, 3-mercapto-1-propanesulfonic acid and the alkali metal and ammonium salts thereof.

Various semiconductor architectures comprising the inventive semiconductors are also conceivable, for example top contact, top gate, bottom contact, bottom gate, or else a vertical construction, for example a VOFET (vertical organic field-effect transistor), as described, for example, in US 2004/0046182.

The layer thicknesses are, for example, from 10 nm to 5 μm in semiconductors, from 50 nm to 10 μm in the dielectric; the electrodes may, for example, be from 20 nm to 1 μm. The OFETs may also be combined to form other components such as ring oscillators or inverters.

A further aspect of the invention is the provision of electronic components which comprise a plurality of semiconductor components, which may be n- and/or p-semiconductors. Examples of such components are field-effect transistors (FETs), bipolar junction transistors (BJTs), tunnel diodes, converters, light-emitting components, biological and chemical detectors or sensors, temperature-dependent detectors, photodetectors such as polarization-sensitive photodetectors, gates, AND, NAND, NOT, OR, TOR and NOR gates, registers, switches, timer units, static or dynamic stores and other dynamic or sequential, logical or other digital components including programmable circuits.

A specific semiconductor element is an inverter. In digital logic, the inverter is a gate which inverts an input signal. The inverter is also referred to as a NOT gate. Real inverter circuits have an output current which constitutes the opposite of the input current. Typical values are, for example, (0, +5V) for TTL circuits. The performance of a digital inverter reproduces the voltage transfer curve (VTC), i.e. the plot of input current against output current. Ideally, it is a staged function, and the closer the real measured curve approximates to such a stage, the better the inverter is. In a specific embodiment of the invention, the compounds of the formula (I) are used as organic n-semiconductors in an inverter.

The inventive compounds of the formula I are also particularly advantageously suitable for use in organic photovoltaics (OPVs).

Organic solar cells generally have a layer structure and generally comprise at least the following layers: anode, photoactive layer and cathode. These layers are generally situated on a substrate customary therefor. The structure of organic solar cells is described, for example, in US 2005/0098726 and US 2005/0224905, which are fully incorporated here by reference.

The invention further provides an organic solar cell comprising at least one compound of the formula I as defined above as a photoactive material.

Suitable substrates for organic solar cells are, for example, oxidic materials (such as glass, ceramic, SiO₂, in particular quartz, etc.), polymers (e.g. polyvinyl chloride, polyolefins such as polyethylene and polypropylene, polyesters, fluoropolymers, polyamides, polyurethanes, polyalkyl(meth)acrylates, polystyrene and mixtures and composites thereof) and combinations thereof.

Suitable electrodes (cathode, anode) are in principle metals (preferably of groups 8, 9, 10 or 11 of the Periodic Table, e.g. Pt, Au, Ag, Cu, Al, In, Mg, Ca), semiconductors (e.g. doped Si, doped Ge, indium tin oxide (ITO), gallium indium tin oxide (GITO), zinc indium tin oxide (ZITO), etc.), metal alloys (e.g. based on Pt, Au, Ag, Cu, etc., especially Mg/Ag alloys), semiconductor alloys, etc. The anode used is preferably a material essentially transparent to incident light. This includes, for example, ITO, doped ITO, ZnO, TiO₂, Ag, Au, Pt. The cathode used is preferably a material which essentially reflects the incident light. This includes, for example, metal films, for example of Al, Ag, Au, In, Mg, Mg/Al, Ca, etc.

The photoactive layer itself comprises at least one, or consists of at least one, layer which has been provided by a process according to the invention and comprises, as an organic semiconductor material, comprises at least one compound of the formula Ia and/or Ib as defined above. In addition to the photoactive layer, there may be one or more further layers. These include, for example,
layers with electron-conducting properties (ETLS, electron transport layers)
layers which comprise a hole-conducting material (hole transport layer, HTL) which must not absorb,
exciton- and hole-blocking layers (e.g. exciton blocking layers, EBLs) which should not absorb, and
multiplication layers.

Suitable exciton- and hole-blocking layers are described, for example, in U.S. Pat. No. 6,451,415.

Suitable materials for exciton blocker layers are, for example, bathocuproin (BCP), 4,4′,4″-tris[3-methylphenyl-N-phenylamino]triphenylamine (m-MTDATA) or poly-ethylenedioxythiophene (PEDOT).

The inventive solar cells may be based on photoactive donor-acceptor heterojunctions. Where at least one compound of the formula I is used as an HTM (hole transport material), the corresponding ETM (exciton transport material) must be selected such that, after excitation of the compounds, a rapid electron transition to the ETM takes place. Suitable ETMs are, for example, C60 and other fullerenes, perylene-3,4;9,10-bis(dicarboximides) (PTCDI), etc. When at least one compound of the formula I is used as an ETM, the complementary HTM has to be selected such that, after excitation of the compound, a rapid hole transition to the HTM takes place. The heterojunction may have a flat (smooth) design (cf. Two layer organic photovoltaic cell, C. W. Tang, Appl. Phys. Lett., 48 (2), 183-185 (1986) or N. Karl, A. Bauer, J. Holzäpfel, J. Marktanner, M. Möbus, F. Stölzle, Mol. Cryst. Liq. Cryst., 252, 243-258 (1994)). The heterojunction may also be designed as a bulk heterojunction or interpenetrating donor-acceptor network (cf., for example, C. J. Brabec, N. S. Sariciftci, J. C. Hummelen, Adv. Funct. Mater., 11 (1), 15 (2001)).

The compounds of the formula I may be used as a photoactive material in solar cells with MiM, pin, pn, Mip or Min structure (M=metal, p=p-doped organic or inorganic semiconductor, n=n-doped organic or inorganic semiconductor, i=intrinsically conductive system composed of organic layers; cf., for example, B. J. Drechsel et al., Org. Eletron., 5 (4), 175 (2004) or Maennig et al., Appl. Phys. A 79, 1-14 (2004)).

The compounds of the formula I can also be used as photoactive material in tandem cells, as described by P. Peumans, A. Yakimov, S. R. Forrest in J. Appl. Phys, 93 (7), 3693-3723 (2003) (cf. patents U.S. Pat. No. 4,461,922, U.S. Pat. No. 6,198,091 and U.S. Pat. No. 6,198,092).

The compounds of the formula I may also be used as photoactive material in tandem cells composed of two or more stacked MiM, pin, Mip or Min diodes (cf. patent application DE 103 13 232.5) (J. Drechsel et al., Thin Solid Films, 451452, 515-517 (2004)).

The layer thicknesses of the M, n, i and p layers are typically from 10 to 1000 nm, preferably from 10 to 400 nm, more preferably from 10 to 100 nm. Thin layers can be produced by vapor deposition under reduced pressure or in inert gas atmosphere, by laser ablation or by solution- or dispersion-processable processes such as spin-coating, knife-coating, casting processes, spraying, dip-coating or printing (e.g. inkjet, flexographic, offset, gravure; intaglio printing, nanoimprinting).

Suitable organic solar cells may, as mentioned above, comprise at least one inventive compound of the formula I as an electron donor (n-semiconductor) or electron acceptor (p-semiconductor). In addition to the compounds of the general formula I, the following semiconductor materials are suitable for use in organic photovoltaics:

Phthalocyanines which are unhalogenated or halogenated. These include metal-free phthalocyanines or phthalocyanines comprising divalent metals or groups containing metal atoms, especially those of titanyloxy, vanadyloxy, iron, copper, zinc, etc. Suitable phthalocyanines are especially copper phthalocyanine, zinc phthalocyanine and metal-free phthalocyanine. In a specific embodiment, a halogenated phthalocyanine is used. These include:

2,6,10,14-tetrafluorophthalocyanines, e.g. copper 2,6,10,14-tetrafluorophthalocyanine and zinc 2,6,10,14-tetrafluorophthalocyanine;

1,5,9,13-tetrafluorophthalocyanines, e.g. copper 1,5,9,13-tetrafluorophthalocyanines and zinc 1,5,9,13-tetrafluorophthalocyanines;

2,3,6,7,10,11,14,15-octafluorophthalocyanine, e.g. copper 2,3,6,7,10,11,14,15-octafluorophthalocyanine and zinc 2,3,6,7,10,11,14,15-octafluorophthalocyanine; hexadecachlorophthalocyanines and hexadecafluorophthalocyanines, such as copper hexadecachlorophthalocyanine, zinc hexadecachlorophthalocyanine, metal-free hexadecachlorophthalocyanine, copper hexadecafluorophthalocyanine, hexadecafluorophthalocyanine or metal-free hexadefluorophthalocyanine.

Porphyrins, for example 5,10,15,20-tetra(3-pyridyl)porphyrin (TpyP), or else tetrabenzoporphyrins, for example metal-free tetrabenzoporphyrin, copper tetrabenzoporphyrin or zinc tetrabenzoporphyrin. Especially preferred are tetrabenzoporphyrins which, like the compounds of the formula (I) used in accordance with the invention, are processed from solution as soluble precursors and are converted to the pigmentary photoactive component by thermolysis on the substrate.

Acenes, such as anthracene, tetracene, pentacene, each of which may be unsubstituted or substituted. Substituted acenes preferably comprise at least one substituent which is selected from electron-donating substituents (e.g. alkyl, alkoxy, ester, carboxylate or thioalkoxy), electron-withdrawing substituents (e.g. halogen, nitro or cyano) and combinations thereof. These include 2,9-dialkylpentacenes and 2,10-dialkylpentacenes, 2,10-dialkoxypentacenes, 1,4,8,11-tetraalkoxypentacenes and rubrene(5,6,11,12-tetraphenylnaphthacene). Suitable substituted pentacenes are described in US 2003/0100779 and U.S. Pat. No. 6,864,396, which are hereby incorporated by reference. A preferred acene is rubrene.

Liquid-crystalline (LC) materials, for example coronenes, such as hexabenzocoronene (HBC—PhC₁₂), coronenediimides, or triphenylenes such as 2,3,6,7,10,11-hexahexylthio-triphenylene (HTT₆), 2,3,6,7,10,11-hexakis(4-n-nonylphenyl)triphenylene (PTP₉) or 2,3,6,7,10,11-hexakis(undecyloxy)triphenylene (HAT₁₁). Particular preference is given to liquid-crystalline materials which are discotic.

Thiophenes, oligothiophenes and substituted derivatives thereof; suitable oligothiophenes are quaterthiophenes, quinquethiophenes, sexithiophenes, α,ω-di(C₁-C₈)-alkyl-oligothiophenes, such as α,ω-dihexylquaterthiophene, α,ω-dihexylquinquethiophene and α,ω-dihexylsexithiophene, poly(alkylthiophenes), such as poly(3-hexylthiophene), bis(dithienothiophenes), anthradithiophenes and dialkylanthradithiophenes such as dihexylanthradithiophene, phenylene-thiophene (P-T) oligomers and derivatives thereof, especially α,ω-alkyl-substituted phenylene-thiophene oligomers.

Also suitable are compounds of the α,α′-bis(2,2-dicyanovinyl)quinquethiophene (DCV5T) type, 3-(4-octylphenyl)-2,2′-bithiophene (PTOPT) type, poly(3-(4′-(1,4,7-tri-oxaoctyl)phenyl)thiophene) (PEOPT) type, poly(3-(2′-methoxy-5′-octylphenyl)thiophene) (POMeOPT) type, poly(3-octylthiophene) (P₃OT) type, poly(pyridopyrazine-vinylene)-polythiophene blends, such as EHH-PpyPz, PTPTB copolymers, BBL copolymers, F₈BT copolymers, PFMO copolymers; see Brabec C., Adv. Mater., 2996, 18, 2884, (PCPDTBT) poly[2,6-(4,4-bis(2-ethylhexyl)-4H-cyclopenta[2,1 b;3,4 b′]dithiophene)-4,7-(2,1,3-benzothiadiazole).

Paraphenylenevinylene and oligomers or polymers comprising paraphenylenevinylene, for example polyparaphenylenevinylene, MEH-PPV (poly(2-methoxy-5-(2′-ethylhexyloxy)-1,4-phenylenevinylene), MDMO-PPV (poly(2-methoxy-5-(3′,7′-dimethyloctyloxy)-1,4-phenylenevinylene)), PPV, CN-PPV (with various alkoxy derivatives).

Phenyleneethynylene/phenylenevinylene hybrid polymers (PPE-PPV).

Polyfluorenes and alternating polyfluorene copolymers, for example with 4,7-dithien-2′-yl-2,1,3-benzothiadiazole; also suitable are poly(9,9′-dioctylfluorene-co-benzothia-diazole) (F₈BT), poly(9,9′-dioctylfluorene-co-bis-N,N′-(4-butylphenyl)bis-N,N′-phenyl-1,4-phenylenediamine (PFB).

Polycarbazoles, i.e. oligomers and polymers comprising carbazole.

Polyanilines, i.e. oligomers and polymers comprising aniline.

Triarylamines, polytriarylamines, polycyclopentadienes, polypyrroles, polyfurans, polysiloles, polyphospholes, TPD, CBP, Spiro-MeOTAD.

Particular preference is given to using, in organic solar cells, a combination of semiconductor materials which comprises at least one inventive compound and a halogenated phthalocyanine.

Rylenes other than the compounds of the formula I used in accordance with the invention. In this context, the term “rylenes” generally refers to compounds having a molecular moiety composed of peri-linked naphthalene units. According to the number of naphthalene units, they may be perylenes (n=2), terrylenes (n=3), quaterrylenes (n=4) or higher rylenes. Accordingly, they may be perylenes, terrylenes or quaterrylenes of the following formula

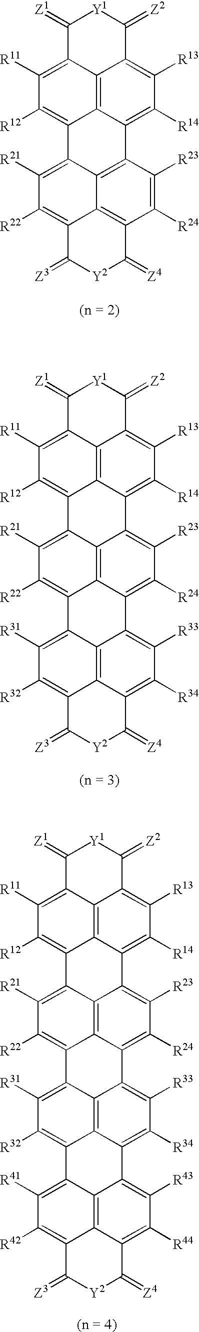

in which

the Rⁿ¹, Rⁿ², Rⁿ³ and Rⁿ⁴ radicals, when n=from 1 to 4, may each independently be hydrogen, halogen or groups other than halogen,

Y¹ is O or NR^{a}, where R^{a} is hydrogen or an organyl radical,

Y² is O or NR^{b}, where R^{b} is hydrogen or an organyl radical,

Z¹, Z², Z³ and Z⁴ are each O,

where, in the case that Y¹ is NR^{a}, one of the Z¹ and Z² radicals may also be NR^{c}, where the R^{a} and R^{c} radicals together are a bridging group having from 2 to 5 atoms between the flanking bonds, and

where, in the case that Y² is NR^{b}, one of the Z³ and Z⁴ radicals may also be NR^{d}, where the R^{b} and R^{d} radicals together are a bridging group having from 2 to 5 atoms between the flanking bonds.

Suitable rylenes are described, for example, in PCT/EP2006/070143, PCT/EP2007/051532 and PCT/EP2007/053330, which are hereby incorporated by reference.

Particular preference is given to using, in organic solar cells, a combination of semiconductor materials which comprises at least one inventive rylene of the formula I.

All aforementioned semiconductor materials may also be doped. In a specific embodiment, in the inventive organic solar cells, the compound of the formula I and/or (if present) a further semiconductor material different therefrom is thus used in combination with at least one dopant. Suitable dopants for use of the compounds I as n-semiconductors are, for example, pyronin B and rhodamine derivatives.

The invention further relates to an organic light-emitting diode (OLED) which comprises at least one inventive compound of the formula I.

Organic light-emitting diodes are in principle formed from a plurality of layers. These include: 1. anode, 2. hole-transporting layer, 3. light-emitting layer, 4. electron-transporting layer and 5. cathode. It is also possible that the organic light-emitting diode does not have all of the layers mentioned; for example, an organic light-emitting diode comprising layers (1) (anode), (3) (light-emitting layer) and (5) (cathode) is likewise suitable, in which case the functions of layers (2) (hole-transporting layer) and (4) (electron-transporting layer) are assumed by the adjacent layers. OLEDs which have layers (1), (2), (3) and (5) or layers (1), (3), (4) and (5) are likewise suitable. The structure of organic light-emitting diodes and processes for their production are known in principle to those skilled in the art, for example from WO 2005/019373. Suitable materials for the individual layers of OLEDs are disclosed, for example, in WO 00/70655. Reference is made here to the disclosure of these documents. Compounds I can be applied to a substrate by deposition from the gas phase by customary techniques, i.e. by thermal evaporation, chemical vapor deposition and other techniques.

The invention is illustrated in detail with reference to the following nonrestrictive examples.

### EXAMPLES

### Example 1

### Preparation of 2-pentafluorophenylethylamine

370 mg of LiAlH₄ are suspended in 10 ml of dry diethyl ether. 1.24 g of AlCl₃ are then dissolved in 6 ml of ether and added rapidly to the suspension. After 5 min, 2.00 g of pentafluorophenylacetonitrile dissolved in 6 ml of ether are slowly added dropwise. After stirring at room temperature for one hour, the remaining LiAlH₄ is quenched cautiously with water, then 16 ml of 6N sulfuric acid and 8 ml of water are added. In a separating funnel, the ether phase is removed and the aqueous phase is extracted by shaking twice with 20 ml each time of ether. Finally, the aqueous phase is brought to pH 11 with KOH pellets while cooling with an ice bath, and the aqueous phase is once again extracted by shaking three times with 30 ml each time of ether. These three organic phases are combined and dried over sodium sulfate, and the solvent is removed under gentle vacuum.

Yield: 1.70 g (0.85 mmol, 85% of theory)

¹H NMR (400 MHz, CDCl₃, TMS):

δ=2.95 (t, 2H, ³J=7.2 Hz), 2.83 (t, 2H, ³J=7.2 Hz)

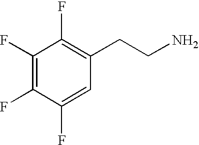

### Example 2

### Preparation of N,N′-bis(2-pentafluorophenylethyl)perylene-3,4:9,10-tetracarboximide

50 mg (0.127 mmol) of perylene-3,4:9,10-tetracarboxylic bisanhydride, 200 mg (0.984 mmol) of 2-pentafluorophenylethylamine and 5 mg of zinc acetate are suspended under argon in 0.7 ml of quinoline, and the mixture is heated to 180° C. overnight. The reaction mixture is then added to 2N HCl and extracted by shaking three times with 100 ml of chloroform. The organic phases are combined, dried over sodium sulfate and purified by column chromatography (CHCl₃). In order to remove last traces of a by-product, the mixture was eluted once again with chloroform/toluene (9/1) on silica gel.

Yield: 15 mg (15% of theory) (red powder)

¹H NMR (400 MHz, CDCl₃, TMS, 55° C., extremely low solubility):

δ=3.25 (t, 4H, ³J=6.8 Hz), 4.53 (t, 4H, ³J=6.6 Hz), 8.67 (m, 8H)

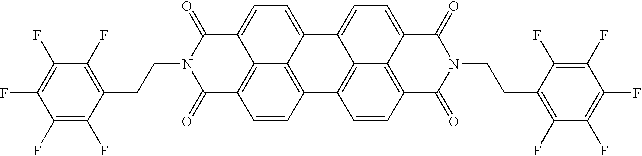

HR-MS (apci (pos-mode, acetonitrile/chloroform:1/1)): calc. m/z C₄₀H₁₇F₁₀N₂O₄ ([M+H]⁺) 779, 1023; found 779, 1025.

Electrochemistry (CH₂Cl₂, 0.1M TBAHFP, vs. ferrocene):

E^{red}_{1/2} (PBI/PBI⁻)=−1.01 V, E^{red}_{1/2} (PBI⁻/PB²⁻)=−1.21 V

Performance results when used in field-effect transistors:

Production of semiconductor substrates by means of deposition from the gas phase

The substrates used were n-doped silicon wafers (2.5×2.5 cm, conductivity<0.004 Ω⁻¹cm) with a thermally deposited oxide layer (300 nm) as the dielectric (area-based capacitance Cᵢ=10 nF/cm²). The coated substrates were cleaned by rinsing with acetone and isopropanol. The semiconductor compounds were PVD deposited on the substrate at defined temperatures (125° C.) with a deposition rate in the range from 0.3 to 0.5 Å/s and a pressure of 10⁻⁶ torr in a vacuum deposition apparatus (Angstrom Engineering Inc., Canada). To measure the charge mobilities of the resulting material, TFTs were provided in top-contact configuration. To this end, source and drain electrodes of channel length 100 μm and a length/width ratio of about 20, by means of photolithography and gas phase deposition, a 60 nm gold layer was deposited onto 4 nm of chromium. The surfaces of the substrates were modified with OTS as described hereinafter or left unmodified. The electrical properties of the OFETs were determined by means of a Keithley 4200-SCS semiconductor parameter analyzer.

Surface Treatment

After the SiO₂-coated wafer had been cleaned by rinsing with acetone and isopropanol, the surface was modified with n-octadecyltriethoxysilane (OTS, C₁₈H₃₇Si(OC₂H₅)₃). To this end, a few drops of OTS (Aldrich Chem. Co.) were placed onto the preheated surface (about 100° C.) in a vacuum desiccator. The desiccator was evacuated and the substrates were kept under vacuum for 5 hours (25 mm Hg). Finally, the substrates were baked at 110° C. for 15 minutes, rinsed with isopropanol and dried in a nitrogen stream.

The results of the testing of the transistor properties are reproduced in table 1.

TABLE 1

| | | N₂ | Air*) |
|---|---|---|---|
| with OTS | μ (cm²/Vs) | 0.33 | 0.31 |
| | Iₒₙ/I_{off} | 4.2 × 106 | 5.4 × 107 |
| | V_{T} (V) | 5.8 | 12.5 |
| without OTS | μ (cm²/Vs) | 0.14 | 0.11 |
| | Iₒₙ/I_{off} | 3.3 × 105 | 3.8 × 105 |
| | V_{T} (V) | 5.8 | 12.5 |

*)relative humidity 50%

1. A compound of the general formula (I) 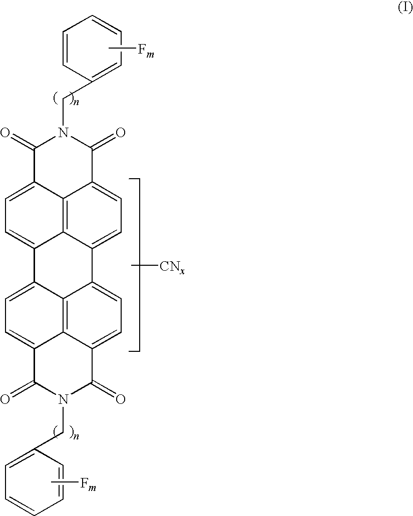
in which
m is 2, 3, 4 or 5,
n is 1, 2 or 3, and
x is 0 or 2.

2. A compound of the general formula I according to claim 1, in which m is 5.

3. A compound of the general formula I according to either of the preceding claims, in which n is 2.

4. A compound of the general formula I according to any one of the preceding claims, in which x is 0.

5. A process for preparing a compound of the general formula I as defined in any one of claims 1 to 4, in which a compound of the general formula II 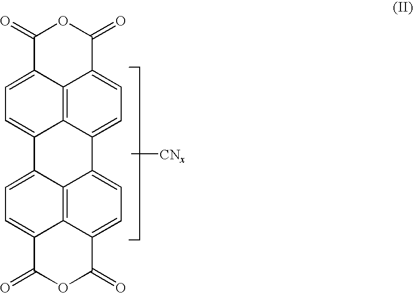
in which x is 0 or 2,
is reacted with an amine of the general formula III 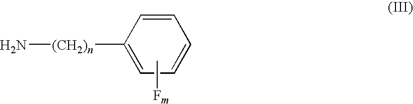
in which
n is 1, 2 or 3, and
m is 2, 3, 4 or 5.

6. A charge transport material, exciton transport material or emitter material, comprising a compound of the general formula I as defined in any one of claims 1 to 4.

7. A semiconductor material, comprising a compound of the general formula I as defined in any one of claims 1 to 4.

8. An organic field-effect transistor, comprising a compound of the general formula I as defined in any of claims 1 to 4.

9. An active material in organic photovoltaics, especially an exciton transport material in excitonic solar cells, comprising a compound of the general formula I as defined in any one of claims 1 to 4.

10. An organic field-effect transistor comprising a substrate having at least one gate structure, a source electrode and a drain electrode and at least one compound of the formula I as defined in any one of claims 1 to 4 as an n-semiconductor.

11. A substrate comprising a multitude of organic field-effect transistors, wherein at least some of the field-effect transistors comprise at least one compound of the formula I as defined in any one of claims 1 to 4 as an n-semiconductor.

12. A semiconductor unit comprising at least one substrate as defined in claim 11.

13. An organic light-emitting diode (OLED) comprising at least one compound of the formula I as defined in any one of claims 1 to 4.

14. An organic solar cell comprising at least one compound of the formula I as defined in any one of claims 1 to 4.
